(19) United States
(12) Patent Application Publication
(10) Pub. No.: US 2011/0207697 A1
(43) Pub. Date: Aug. 25, 2011

(54) XANTHOHUMOL COMPOSITIONS AND METHODS FOR TREATING SKIN DISEASES OR DISORDERS

(75) Inventors: Mitsunori Ono, Lexington, MA (US); Naoto Yamaguchi, Bethesda, MD (US); Keiko Yamaguchi, Bethesda, MD (US)

(73) Assignee: Betal, LLC, Washington, DC (US)

(21) Appl. No.: 12/937,237

(22) PCT Filed: Apr. 10, 2009
(86) PCT No.: PCT/US09/02266

§ 371 Date: May 2, 2011

Related U.S. Application Data
(60) Provisional application No. 61/044,231, filed on Apr. 11, 2008.

(51) Int. Cl. A61K 31/724; A61K 8/73; A61K 31/122; A61K 8/35; A61P 17/00; A61P 31/04; A61Q 19/08

(52) U.S. Cl. 514/58; 514/685

(57) ABSTRACT

The present invention features therapeutic and prophylactic compositions comprising xanthohumol and/or xanthohumol/cyclodextrin complexes and methods of using such compositions for treating a skin condition, disease, or disorder.

### CROSS-REFERENCE TO RELATED APPLICATION

This application claims the benefit of the following U.S. Provisional Application No. 61/044,231, filed on Apr. 11, 2008, the entire contents of which are incorporated herein by reference.

### BACKGROUND OF THE INVENTION

### Background

The skin is the largest organ of the body. It is composed of two layers, the dermis (the thicker lower layer) and the epidermis (the upper surface). The epidermis serves primarily as a barrier against pathogens, environmental chemicals, and ultraviolet (UV) light, while the dermis provides mechanical support and flexibility; it is comprised primarily of collagen bundles and elastic fibers. Trauma and numerous skin diseases or disorders negatively affect the health and appearance of skin. Compositions that promote skin health, that treat or prevent skin diseases and disorders, and that improve the appearance and condition of skin are desired.

### SUMMARY OF THE INVENTION

As described below, the present invention features compositions featuring xanthohumol and methods of using such compositions for treating a skin condition, disease, or disorder.

In one aspect, the invention generally provides a composition for treating or preventing a skin disease, disorder, or condition the composition comprising an effective amount of a xanthohumol/cyclodextrin complex in a pharmaceutically acceptable excipient. In one embodiment, the skin disease, disorder or condition is selected from the group consisting of acne, atopic dermatitis, contact dermatitis, eczema, rosacea, seborrhea, psoriasis, wrinkles, skin thinning, surface blood vessels, loss of elasticity, hyperpigmentation, photodamage, aging, and loss of subcutaneous fat layer.

In another aspect, the invention provides a composition for enhancing the appearance of skin, the composition comprising an effective amount of a xanthohumol or a xanthohumol/cyclodextrin complex in a pharmaceutically acceptable excipient.

In yet another aspect, the invention provides a composition for treating or preventing a skin disease or disorder, the composition comprising an effective amount of a xanthohumol or xanthohumol/cyclodextrin complex in a pharmaceutically acceptable excipient.

In yet another aspect, the invention provides a personal care composition comprising an effective amount of xanthohumol, a xanthohumol/cyclodextrin complex or another hop derivative in a cosmetically acceptable excipient. In one embodiment, the xanthohumol is about 0.001% to about 10% (e.g., about 0.01% to about 5%, 0.01% to about 1%, 0.01%, 0.05%, 0.1%, 1%, 2%, 3%, 4%, 5%) of the composition.

In yet another aspect, the invention provides a nutraceutical composition comprising an effective amount of xanthohumol or a xanthohumol/cyclodextrin complex in an edible carrier.

In still another aspect, the invention provides a non-alcoholic food product composition comprising xanthohumol or a xanthohumol/cyclodextrin complex. In one embodiment, the food product is selected from the group consisting of milk, tea, soft drink, juice, coffee, seasoning, cereal, water, yogurt, cookies, chewing gum, chocolate, and soup.

In yet another aspect, the invention provides a dietary supplement composition comprising an effective amount of xanthohumol or a xanthohumol/cyclodextrin complex.

In yet another aspect, the invention provides a method for treating or preventing a skin disease or disorder (e.g., acne, dermatitis, atopic dermatitis, contact dermatitis, seborrheic dermatitis, eczema, psoriasis, rosacea, wounding, and scarring) in a subject, the method comprising administering to the subject an effective amount of xanthohumol or a xanthohumol/cyclodextrin complex.

In yet another aspect, the invention provides a method for treating or preventing a skin disease, disorder or condition in a subject, the method comprising administering to the subject an effective amount of a xanthohumol/cyclodextrin complex. In one embodiment, the skin disease, disorder or condition is selected from the group consisting of acne, atopic dermatitis, contact dermatitis, eczema, rosacea, seborrhea, psoriasis, wrinkles, skin thinning, surface blood vessels, loss of elasticity, hyperpigmentation, photodamage, aging, and loss of subcutaneous fat layer.

In yet another aspect, the invention provides a method for enhancing the appearance of skin in a subject, the method comprising administering to the subject an effective amount of xanthohumol or a xanthohumol/cyclodextrin complex. In one embodiment, the skin's appearance is enhanced by reducing the appearance of fine lines, wrinkles, sagging, or hyperpigmentation. In another embodiment, the skin's appearance is enhanced by increasing skin smoothness, firmness, or elasticity.

In yet another aspect, the invention provides a method for ameliorating acne in a subject in need thereof, the method comprising administering to the subject an effective amount of xanthohumol or a xanthohumol/cyclodextrin complex. In one embodiment, the method reduces inflammation and matrix remodeling associated with severe acne. In another embodiment, the method reduces bacteria present on skin, reduces oxidative damage, and reduces inflammation. In yet another embodiment, the method reduces the survival or proliferation of a bacteria selected from the group consisting of Propionibacterium acnes, Staphylococcus epidermidis, Staphylococcus aureus, Kocuria rhizophila and Staphylococcus pyogenes. In another embodiment, the method provides an activity selected from the group consisting of singlet oxygen-quenching activity, antioxidant activity, anti-inflammatory activity where inflammation is caused by oxidative damage, and total oxygen radical absorbance activity.

In yet another aspect, the invention provides a method for inhibiting collagenase activity, oxidative damage, and/or inflammation in a cell, the method comprising contacting the cell with an effective amount of xanthohumol or a xanthohumol cyclodextrin complex.

In yet another aspect, the invention provides a pharmaceutical pack comprising xanthohumol or a xanthohumol/cyclodextrin complex formulated in individual dosage amounts.

In yet another aspect, the invention provides a method of preparing a xanthohumol/cyclodextrin complex comprising

(a) providing a mixture of xanthohumol and gamma cyclodextrin;

(b) adjusting the pH of the mixture to 10-12, thereby providing for xanthohumol/cyclodextrin complex formation;

(c) re-adjusting the pH to 6-9 to allow precipitation of the xanthohumol/cyclodextrin complex; and

(d) recovering the complex.

In yet another aspect, the invention provides a composition made by the method of claim 50, said composition comprising a xanthohumol/cyclodextrin complex, wherein the composition comprises less than about 5% isoxanthohumol.

In yet another aspect, the invention provides a method for treating or preventing a skin disease, disorder or condition in a subject, the method comprising contacting the subject with the composition of claim 51, wherein the composition comprises less than about 0.5-3% isoxanthohumol. In one embodiment, the skin disease or disorder acne, atopic dermatitis, contact dermatitis, eczema, rosacea, seborrhea, psoriasis, wrinkles, skin thinning, surface blood vessels, loss of elasticity, hyperpigmentation, photodamage, aging and loss of subcutaneous fat layer.

In various embodiments of the above aspects or any other aspect of the invention described herein, the composition has anti-collagenase activity, enhances collagen synthesis or deposition in a cell, has anti-bacterial activity, has oxygen radical absorbance capacity and/or singlet oxygen absorbance capacity. In other embodiments of the invention, the composition is formulated for oral or topical administration. In various embodiments, the composition features an effective amount of xanthohumol that is 0.1 μg-200 mg (e.g., 0.25 μg, 0.5 μg, 10 μg, 20 μg, 30 μg, 40 μg, 50 μg, 100 μg, 500 μg, or 1 mg, 5 mg, 10 mg, 25 mg, 50 mg, 100 mg, 125 mg, 150 mg, 175 mg or 200 mg per dose per day. In still other embodiments of the above aspects, the xanthohumol is 3′-[3,3-dimethyl allyl]-2′,4′,4-trihydroxy-6′-methoxychalcone) or a prenylated chalcone derived from hops. In still other embodiments, the xanthohumol is selected from the group consisting of xanthoangelol, xanthoangelol F, 4-hydroxyderricin, 4-O-methylxanthohumol, isobavachalcone, xanthoangelol H, xanthogalenol, desmethoxyxanthohumol, 5′-prenylxanthohumol, tetrahydroxanthohumol, 2′,4′,6′,4-terahydroxy-3′-C-geranylchalcone, dehydrocycloxanthohumol, 4-O-5′-C-diphenylxanthohumol, 4′-O-methylxanthohumol, and a xanthohumol metabolite. In still other embodiments, the cyclodextrin is alpha-cyclodextrin, beta-cyclodextrin, or gamma-cyclodextrin. In still other embodiments, the cyclodextrin compound is gamma-cyclodextrin. In still other embodiments of an invention defined herein, the cyclodextrin is selected from the group consisting of hydroxypropyl-beta-cyclodextrin, sulfobutyl ether-beta-cyclodextrin, heptakis(2,6-di-O-methyl)-beta cyclodextrin, C₁₋₂₄-alkyl-gamma-cyclodextrin, and C₁₋₂₄-hydroxyalkyl-gamma-cyclodextrin. In still other embodiments, the xanthohumol and the cyclodextrin have a molar ratio of 2:1 to 1:4 (e.g., 1:2, 1:3) in the complex. In still other embodiments of the above aspects of any other aspect of the invention delineated herein, the composition comprises or consists essentially of 0.01-30%, 0.05-20%, or 0.1-10% (0.01, 0.05, 0.1, 0.2, 0.3, 0.5, 1, 2, 3, 4, 5, 10, 15, 20, 25, or 30%) by weight xanthohumol. In various other embodiments of the above aspects, the xanthohumol/cyclodextrin complex is at least about 5-10 (e.g., 1, 2, 3, 4, 5, 6, 7, 8, 9, 10) times more soluble in water than xanthohumol alone. In still other embodiments, the composition comprises less than about 0.05, 1%, 2%, 3%, 4%, or 5% isoxanthohumol. In still other embodiments, the composition comprises less than about 3-5% isoxanthohumol after 3-6 months storage. In still other embodiments, the composition is virtually devoid of isoxanthohumol, a cyclodextrin/isoxanthohumol complex or comprises less than about 10% of a cyclodextrin/isoxanthohumol complex. In still other embodiments of the invention, the composition further contains a therapeutic agent selected from the group consisting of a vitamin, an antifungal agent, a self-tanning agent, an anti-microbial agent, an anti-inflammatory agent, and an insect repellent. In still other embodiments of the above aspects of any other aspect of the invention delineated herein, the xanthohumol is about 0.001% to about 10% (e.g., about 0.01% to about 5%, 0.01% to about 1%, 0.01%, 0.05%, 0.1%, 1%, 2%, 3%, 4%, 5%) of the composition.

The invention provides compositions comprising xanthohumol. Compositions and articles defined by the invention were isolated or otherwise manufactured in connection with the examples provided below. Other features and advantages of the invention will be apparent from the detailed description, and from the claims.

### DEFINITIONS

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “ameliorate” is meant decrease, suppress, attenuate, diminish, arrest, or stabilize the development or progression of a disease.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “cosmetic composition” is meant a product that is intended to be applied to the human body for cleansing, beautifying, or altering the appearance. Such compositions typically provide non-therapeutic benefits and are not regulated as pharmaceuticals. Cosmetic compositions may be incorporated into pharmaceutical compositions to provide cosmetic benefits (e.g., products that treat skin diseases, but also have cosmetic benefits).

By “cosmetic benefit” is meant a desirable change in the appearance of skin that results from the administration of a personal care composition.

By “cyclodextrin” is meant a cyclic oligosaccharide comprising glucose monomers arranged in a toroidal shape or a derivative thereof. Exemplary cyclodextrins include α, β, and γ cyclodextrin, hydroxypropyl-beta-cyclodextrin, sulfobutyl ether-beta-cyclodextrin, heptakis(2,6-di-O-methyl)-beta cyclodextrin, C₁₋₂₄-alkyl-gamma-cyclodextrin, and C₁₋₂₄-hydroxyalkyl-gamma-cyclodextrin.

“Detect” refers to identifying the presence, absence or amount of the object to be detected.

By “disease” is meant any pathological condition that damages or interferes with the normal function of a cell, tissue, or organ.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “isolated” is meant free from the components that naturally accompany the isolated product. In one embodiment, an isolated xanthohumol is at least about 25%, 35%, 50%, 75%, or 100% free of other components typically found in hops.

By “marker” is meant any protein or polynucleotide having an alteration in expression level or activity that is associated with a disease or disorder.

By “reference” is meant a standard or control condition.

By “skin disease” is meant a pathology effecting the epidermis. Exemplary skin diseases include, but are not limited to, acne, dermatitis (e.g., atopic dermatitis, contact dermatitis, seborrheic dermatitis), eczema, psoriasis, rosacea, wounding, and scarring. In one embodiment, the effects of aging and photodamage are specifically excluded from the term “skin disease.” In another embodiment, the effects of aging and photodamage are embraced by the term “skin condition,” which refers to epidermal tissue damage associated with the effects of normal aging or sun exposure.

By “makeup composition” is meant a preparation that is used to beautify, care for, maintain, or augment the appearance of a subject. Exemplary makeup compositions include, but are not limited to, color cosmetics, such as lipsticks, lip liners, eye shadows, eyeliners, rouges, face powders, foundations, and blushes.

As used herein, “obtaining” as in “obtaining an agent” includes synthesizing, purchasing, or otherwise acquiring the agent.

By “personal care composition” is meant a product for application to the skin for the purposes of improving, cleaning, beautifying, treating, and/or caring for the skin or an associated tissue.

By “skin care composition” is meant a composition that is applied to skin to remove or minimize acne, wrinkles, reduce pigmentation, soften, smooth, or cleanse skin, or improve skin tone.

By “therapeutic composition” is meant a material that is used to ameliorate or treat a disease or disorder.

By “xanthohumol” is meant a prenylated chalcone derived from hops or a derivative thereof. Exemplary xanthohumols include but are not limited to 3′-[3,3-dimethyl allyl]-2′,4′,4-trihydroxy-6′-methoxychalcone), xanthoangelol, xanthoangelol F, 4-hydroxyderricin, 4-O-methylxanthohumol, isobavachalcone, xanthoangelol H, xanthogalenol, desmethoxyxanthohumol, 5′-prenylxanthohumol, tetrahydroxanthohumol, 2′,4′,6′,4-terahydroxy-3′-C-geranylchalcone, dehydrocycloxanthohumol, 4-O-5′-C-diphenylxanthohumol, 4′-O-methylxanthohumol, and xanthohumol degradation products. In other embodiments,

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

### BRIEF DESCRIPTION OF THE DRAWINGS

FIG. 1 shows structures of hop components (1-7).

FIG. 2 provides a graphical representation of antioxidant activities of hop components in six assay categories.

### DETAILED DESCRIPTION OF THE INVENTION

The invention provides therapeutic and prophylactic compositions comprising xanthohumol and methods of using such compositions for the treatment of skin diseases.

The invention is based, at least in part, on the discovery that various hop extracts, including xanthohumol, inhibit the bacteria that cause acne. Seven naturally derived components from the hops plant (Humulus lupulus L.) extracts were tested for evaluation of biological activities affecting acne vulgaris. Five strains, Propionibacterium acnes, Staphylococcus epidermidis, Staphylococcus aureus, Kocuria rhizophila and, Staphylococcus pyogenes, were selected as the main acne-causing bacteria. Hop extracts xanthohumol and the lupulones showed strong inhibitory activities against all of the strains. Although hydrogenated derivatives did not show the same level of activity, naturally occurring xanthohumol, humulones, and lupulones all showed moderate to strong anticollagenase inhibitory activities. Antioxidant capacity was also evaluated with seven different methods based on different reactive oxygen species. Xanthohumol showed the highest activity in total oxygen radical absorbance capacity, as well as singlet oxygen absorbance capacity.

### Acne

Acne vulgaris is one of the most common skin diseases affecting children and adolescents. The pathogenesis of acne is multifactorial, with primary accompanying features being increased sebum production during early puberty; the proliferation of bacteria, such as P. acnes, S. epidermidis, S. aureus, K. rhisophila, and S. pyogenes causing primary or secondary skin infections; and abnormal follicular keratinization and inflammation. P. acnes and S. epidermidis are pus-forming organisms that trigger inflammation in acne. S. pyogenes, K. rhizophila, and S. aureus are often isolated from patients with clinical symptoms of acne.

Acne skin-care preparations containing antibiotics, which are useful for treatment of mild to moderate inflammatory acne, partially exert their beneficial effects by decreasing the follicular population of P. acnes. However, the widespread use of antibiotics in dermatological treatments has led to the development of drug-resistant P. acnes strains. Benzoyl peroxide(BP) is widely used as a topical treatment for acne because of its antibacterial activity. However, it has been reported that BP generates free radicals in the skin. In 1995 the US Federal Drug Administration changed BP from a Category I(safe) to a Category III (safety is uncertain) ingredient based on information that raised a safety concern relating to BP as a tumor promoter in mice. Topical retinoids are important tools in the management of acne because they act against comedones and microcomedones, and have direct anti-inflammatory effects. The retinoids approved for acne treatment include tretinoin (all-trans retinoicacid) and isotretinoin (13-cisretinoicacid), as well as the synthetic third-generation polyaromatic retinoids adapalene and tazarotene. Retinoids in fact account for roughly half of the US prescription acne medicine market. Although the minimal systemic availability of topical retinoid creams has been confirmed, teratogenicity remains a worrisome effect of the retinoids. To overcome the potential risk of adverse effects and antibiotic resistance from prescription medications, alternative treatments that lack such side-effects would provide significant benefits. The present invention, which comprises hops extracts, which are generally regarded as safe provides an advantage over conventional acne remedies.

### Xanthohumol

Xanthohumol (3′-[3,3-dimethyl allyl]-2′,4′,4-trihydroxy-6′-methoxychalcone) is a prenylated chalcone derived from hops (Humulus lupulus L.), specifically the female flowers of the hop plant, which are used in the brewing industry to add flavor and bitterness to beer. Xanthohumol and related prenylated flavonoids (e.g., 2′,4′6′,4-tetrahydroxy-3′-prenylchalcone; 2′,4′,6′,4-tetrahydroxy-3′ geranylchalcone; dehydrocycloxanthohumol; isoxanthohumol), have a variety of biological activities that indicate that such compounds can act as chemopreventive or chemotherapeutic agents. For example, xanthohumol has antioxidant activity (Gerhauser, et al., Molecular Cancer Therapeutics 1:959-969, 2002), anti-proliferative activity (Miranda et al., Food Chem. Toxicol. 37:271-85; 1999; Goto et al., Cancer Letters 219:215-22; Gerhauser, et al., Molecular Cancer Therapeutics 1:959-969, 2002), antiestrogenic activity (Gerhauser, et al., Molecular Cancer Therapeutics 1:959-969, 2002), anti-inflammatory activity (Gerhauser, et al., Molecular Cancer Therapeutics 1:959-969, 2002), and cytotoxic activities (Miranda et al., Food Chem. Toxicol. 37:271-85; 1999). In addition, xanthohumol is reported to inhibit diacylglycerol acyltransferase (Tabata et al., Phytochemistry 46:683-687, 1997).

Xanthohumol isomerizes to form isoxanthohumol, particularly when compositions containing xanthohumol are heated or stored. As reported herein, xanthohumol may be used alone or, if desired to reduce susceptibility to isomerization, xanthohumol may be complexed with cyclodextrin.

### Cyclodextrin

The enzymatic degradation of starch by cyclodextrin-glycosyltransferase (CGT) produces cyclic oligomers, termed cyclodextrins. Cyclodextrins are non-reducing, crystalline, water soluble, cyclic oligosaccharides that consist of glucose monomers arranged in a toroidal shape, which forms a tight conical cylinder having a hydrophilic exterior (due to the presence of hydroxyl radicals) and a hydrophobic interior cavity. The hydrophobic internal cavity provides for the formation of inclusion complexes with a variety of “guest” hydrophobic molecules (e.g. aromatics, alcohols, halides, fatty acids, esters). Naturally occurring cyclodextrins include α (6 sugar units), β (7 sugar units) and γ (8 sugar units) cyclodextrins.

Cyclodextrins can be modified by various procedures, such as substituting one or more hydrogen atoms in the primary and/or secondary hydroxyl groups. Chemically modified cyclodextrins exhibit substantially increased aqueous solubility while retaining the ability to form inclusion complexes. Cyclodextrin inclusion is a molecular phenomenon in which at least one guest molecule interacts with the cavity of a cyclodextrin molecule to form a stable association. Depending on the molecular weight of the guest, more than one guest molecule may fit into the cavity. Likewise, high molecular weight molecules may bind more than one Cyclodextrin molecule. Therefore a 1 to 1 molar ratio between the guest and the cyclodextrin may not be achieved. Cyclodextrins form inclusion complexes with a broad range hydrophobic molecules. Complex formation may enhance the aqueous solubility of poorly soluble compounds and enhance the stability of agents susceptible to deterioration.

### Xanthohumol/Cyclodextrin Complexes

Cyclodextrin selectively complexes with xanthohumol, but not isoxanthohumol. The xanthohumol/cyclodextrin complex is highly water-soluble and less likely to convert to isoxanthohumol than xanthohumol alone. In this complex (a “host-guest complex”), one or more (e.g., 1, 2, 3, 4) molecules of cyclodextrin forms a toroid structure (i.e., a hollow truncated cone having an interior hydrophobic cavity) comprising one or more molecules (e.g., 1, 2, 3, 4) of xanthohumol.

Xanthohumol/cyclodextrin complexes can be prepared as described herein. Briefly, a composition (e.g., a hop extract or spent hops) comprising xanthohumol is mixed with a cyclodextrin and water or a water miscible solvent to form a mixture. The pH of the mixture is adjusted to 10-12 providing for complex formation between the cyclodextrin and the xanthohumol. The complex is recovered using any method known in the art, such as by collecting the mixture containing the complex or, spray-drying the mixture to obtain a complex-containing powder. In one embodiment, insoluble materials are removed, and the mixture containing the complex is acidified to reach a pH value of 6-9, providing for complex precipitation. The mixture is then maintained at a suitable temperature (e.g., room temperature) for a sufficient period of time (e.g., 2 hr, 6 hr, or 12 hr) to provide for optimal precipitation. The precipitated complex is then collected by any means known in the art, such as centrifugation or filtration. If desired, the precipitate is washed with suitable solvents and dried.

In the method described above, when spent hops are used as the substance containing a xanthohumol compound, they are dispersed in water together with a cyclodextrin compound to form a mixture. The water-insoluble materials present in the spent hops are removed by any means known in the art, such as filtration or centrifugation. This step may be carried out before or after the pH is adjusted to 10-12. When a xanthohumol compound and a cyclodextrin compound are used as the starting materials in the aforementioned method, they are dissolved together in water or a water miscible solvent to form a solution. Alternatively, the xanthohumol compound is dissolved in water or a water miscible solvent first and then mixed with an aqueous solution containing the cyclodextrin compound to form a solution. The solution is then adjusted to pH 10-12. Xanthohumol/cyclodextrin compounds are then precipitated as described above.

As described herein, cyclodextrin selectively forms a complex with xanthohumol, but fails to form a complex with isoxanthohumol or forms a reduced level of such complexes. For example, a composition of the invention comprises less than about 0.5%-10% (e.g., 10%, 7%, 5%, 3%, 1%, 0.5% cyclodextrin/isoxanthohumol complexes. Such complexes may be formulated for delivery by any method known in the art. In one embodiment, a cyclodextrin/xanthohumol complex is formulated for topical delivery. The topical formulation can contain 0.01-5% (e.g., 0.01-1% or 0.05-1%) by weight the xanthohumol compound (e.g., xanthohumol), and optionally, further contain another active agent (e.g., a vitamin, an antifungal agent, a self-tanning agent, an anti-microbial agent, an anti-inflammatory agent, or an insect repellent).

In another embodiment, a xanthohumol or xanthohumol-cyclodextrin complex-containing composition described herein can also be formulated for other suitable routes of administration, e.g., oral administration.

### Skin Diseases And Disorders

The invention provides compositions comprising xanthohumol, or other hops extracts for the treatment of skin diseases and disorders. Skin diseases and disorders include skin alterations associated with acne, aging, dermatitis (e.g., atopic dermatitis, contact dermatitis, seborrheic dermatitis), eczema, photodamage, psoriasis, rosacea, wounding, and scarring. In one embodiment, skin photodamage relating to exposure to UV light is excluded. Existing therapies for such disorders are often inadequate or are associated with adverse side effects. Because the combinations of the invention are derived from natural hops, they are advantageously safe and non-toxic relative to synthetic compounds.

Aging

As the skin ages, changes in the structure and composition of collagen and elastin cause changes in the thickness of the dermis. Aging skin is characterized by a number of undesirable changes including tissue atrophy, coarseness, wrinkling, drying, changes in pigmentation or surface blood vessels, reduced elasticity, and premalignant/malignant neoplasms. Aging skin appears thinner, paler, and more translucent. Large pigmented spots (called age spots, liver spots, or lentigos) often appear. Changes in the connective tissue reduces the skin's strength and elasticity. Because aging skin is thinner, more fragile and has lost the protective subcutaneous fat layer, aging skin is at higher risk for injury. In addition, aging skin repairs itself more slowly than younger skin. Wound healing is significantly slower in the elderly and precancerous neoplasms occur with greater frequency in aging skin. In addition, advanced age is associated with a breakdown of the epithelial barrier of the skin, which enables invasion of tissues by pathogenic organisms.

Dermatitis

Dermatitis is an inflammation of the skin caused by internal or external factors, including food allergies or sensitivities or contact with an irritating substance. In contact dermatitis, for example, contact with an irritant or allergen results in acute inflammation. Treatment typically involves the application of topical corticosteroid medications to reduce inflammation. Severe cases of contact dermatitis may require systemic treatment with corticosteroids to reduce inflammation.

Some forms of dermatitis are chronic. Seborrheic dermatitis is a skin condition characterized by loose, greasy or dry, white to yellowish scales, with or without associated reddened skin. Seborrheic dermatitis may involve the skin of the scalp, eyebrows, eyelids, nasolabial creases, lips, behind the ears, in the external ear, and the skin of the trunk, particularly over the sternum and along skin folds. Treatment often involves the topical application of shampoos or lotions containing selenium, ketoconazole, or corticosteroids.

Atopic dermatitis is another chronic dermatitis. Atopic dermatitis is a chronic inflammatory skin disease associated with cutaneous hyperreactivity to environmental triggers. Many individuals that suffer from asthma also suffer from atopic dermatis. Skin affected by atopic dermatitis is extremely itchy. Scratching of lesions results in swelling, cracking, “weeping” clear fluid, and finally, crusting and scaling. Atopic dermatitis has a complex etiology involving the activation of immunologic and inflammatory pathways. Therapy for atopic dermatitis typically includes the application of topical corticosteroids. Side effects of repeated or long-term use of topical corticosteroids can include thinning of the skin, infections, growth suppression (in children), and stretch marks. Given these adverse side-effects, the use of corticosteroids is not typically recommended during periods of remission, and atopic dermatitis remains a chronic disease for which a safe and effective treatment is required.

Psoriasis

Psoriasis is a chronic inflammatory skin disorder affecting 1-2% of the general population. The characteristic lesion of psoriasis is a sharply demarcated erythematous papule containing hyperproliferating skin cells and infiltrating neutrophils, monocytes, and T lymphocytes. In psoriasis, activated T cells end up in the skin where they trigger an immune response that results in the release of inflammatory cytokines, such as tumor necrosis factor alpha (TNFalpha), interferon-gamma, interleukin (IL)-2, IL-6, IL-8, and IL-12, pro-inflammatory cytokines that are thought to be involved in the pathogenesis of several inflammatory and autoimmune diseases, including atopic dermatitis and psoriasis. These changes result in epidermal hyperplasia and cutaneous inflammation (Gottlieb, J. Immunol. 2005 Aug. 15; 175(4):2721-9).

Many patients experience significant adverse side effects from currently available treatments, including development of skin cancers in nonlesional skin, which is associated with psoralen and UV-A light therapy, abnormal renal function, which is associated with cyclosporin A therapy, and liver abnormalities, which are associated with methotrexate therapy. In contrast to existing therapies for psoriasis, compositions of the invention are naturally safe.

Rosacea

Rosacea is a chronic skin condition linked to inflammatory processes for which no efficacious therapy currently exists.

Accordingly, the present invention provides methods of treating skin disease and/or disorders or symptoms thereof which comprise administering a therapeutically effective amount of a pharmaceutical composition comprising a compound of the formulae herein to a subject (e.g., a mammal such as a human). Thus, one embodiment is a method of treating a subject suffering from or susceptible to a disease or disorder or symptom thereof. The method includes the step of administering to the mammal a therapeutic amount of a compound herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which inflammation, collagenase activity, oxidative damage, or a bacteria (e.g., Propionibacterium acnes, Staphylococcus epidermidis, Staphylococcus aureus, Kocuria rhizophila and, Staphylococcus pyogenes) may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with oxidative stress, inflammation, collagenase activity, or a skin disorder, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Hop Derivatives

A hop derivative is a compound that occurs naturally in a hop plant (Humulus lupulus) or is chemically derived (either through natural biosynthetic processes (e.g., living organism metabolism (e.g., mammal, plant, bacteria)) or by synthetic processes using human intervention (e.g., chemical synthesis). Compositions of the invention include one or more compounds derived from hops. Of particular interest are hop polyphenols, including but not limited phenolic acids, prenylated chalcones, flavonoids, catechins, proanthocyanidins, xanthohumol, and isoxanthohumol. When hops are extracted with pure ethanol, methanol, or ethanol or methanol/water mixtures of a high ethanol content of (e.g., 90% by weight of ethanol), virtually all relevant hop constituents, including xanthohumol, are extracted. See, for example, EP-B1-0 057 435. After removing the solvent (ethanol, methanol, or mixtures thereof), a crude extract is obtained. This crude extract can be separated into a polar fraction and a non-polar fraction containing the xanthohumol. Phase separation can be accelerated and/or completed by centrifuging the crude extract. The non-polar fraction containing the xanthohumol is obtained after phase separation. Approximately 80% of the constituents may be extracted from the ethanol extract using supercritical CO₂, for example, as described in EP-A1-0 320 813. Alternative methods for extracting xanthohumol are provided, for example, at U.S. Pat. No. 6,867,332. Plant extracts are often used for the purification of compounds from plants (e.g., hops). An extract can be prepared by drying and subsequently cutting or grinding the dried material. The term “extract” refers to a concentrated preparation of the essential constituents of a plant, such as hops. Typically, an extract is prepared by drying and powderizing the plant. Optionally, the plant, the dried plant or the powderized plant may be boiled in solution. The extract may be used in liquid form, or it may be mixed with other liquid or solid herbal extracts. Alternatively, the extract may be obtained by further precipitating solid extracts from the liquid form. The extraction process may then be performed with the help of an appropriate choice of solvent, typically ethanol/water mixture, methanol, butanol, iso-butanol, acetone, hexane, petroleum ether or other organic solvents by means of maceration, percolation, repercolation, counter-current extraction, turbo-extraction, or by carbon-dioxide supercritical (temperature/pressure) extraction. The extract may then be further evaporated and thus concentrated to yield by means of air drying, spray drying, vacuum oven drying, fluid-bed drying or freeze-drying, the extract product.

Numerous methods are available for the chemical synthesis of xanthohumol or cyclodextrin. Such compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); L. Paquette, ed., Encyclopedia of Reagents for Organic Synthesis, John Wiley and Sons (1995); and M. Verzele and D. De Keukeleire, Chemistry and Analysis of Hop and Beer Bitter Acids, Elsevier: Amsterdam (1991), and subsequent editions thereof. Chemically synthesized xanthohumol can be separated from a reaction mixture and further purified by a method such as column chromatography, high pressure liquid chromatography, or recrystallization. As can be appreciated by the skilled artisan, further methods of synthesizing the compounds herein will be evident to those of ordinary skill in the art. Additionally, the various synthetic steps may be performed in an alternate sequence or order to give the desired compounds.

The compounds of this invention may contain one or more asymmetric centers and thus occur as racemates and racemic mixtures, single enantiomers, enantiomer mixtures, individual diastereomers and diastereomeric mixtures. All such isomeric forms of these compounds are expressly included in the present invention. The compounds of this invention may also be represented in multiple tautomeric forms, in such instances, the invention expressly includes all tautomeric forms of the compounds described herein. All such isomeric forms of such compounds are expressly included in the present invention. All crystal forms of the compounds described herein are expressly included in the present invention. As used herein, the compounds of this invention, including the compounds of formulae described herein, are defined to include derivatives. Derivatives include compounds of the invention that are modified by appending appropriate functionalities to enhance desired properties.

Acceptable salts of the compounds of this invention include those derived from acceptable inorganic and organic acids and bases. Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic acid, may be employed in the preparation of salts useful as intermediates in obtaining the compounds of the invention and their acceptable acid addition salts. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds disclosed herein. Water or oil-soluble or dispersible products may be obtained by such quaternization.

The ratio of xanthohumol to cyclodextrin ranges between about 1:1 and 1:10. In another embodiment, the ratio of xanthohumol to cyclodextrin ranges between about 10:1 and 1:1. In other embodiments of these ratios include 1:2, 1:3, 1:4, 1:5, 1:6, 1:7, 1:8, 1:9, and 1:10. In preferred embodiments, a preparation of the invention includes between 1 and 95% (e.g., 1, 2, 3, 4, 5, 6, 7, 8, 9, 10, 25, 75, 80, 90, or 95%) of a xanthohumol/cyclodextrin complex in a carrier or diluent. Alternatively, such preparations contain from about 20% to about 80% of a xanthohumol/cyclodextrin complex. Compositions containing xanthohumol are manufactured by ordinary methods. Xanthohumol/cyclodextrin complexes suitable for addition to products can be formulated as ordinary tablets, capsules, solids, liquids, emulsions, slurries, fine granules or powders, which are suitable for administration to products during their preparation, following preparation but prior to storage, or at any time prior to their sale to a vendor or consumer. Lower or higher amounts than those recited above may be required. Specific dosage and treatment regimens are determined empirically as described herein.

### Compound Derivatives

Compositions of the invention include xanthohumol and xanthohumol/cyclodextrin complexes. These compositions include both the compounds themselves and their derivatives (e.g., sugar derivatives, metabolic derivatives, prodrugs, derivatives by isomerization, oxidization product, and reduction product). Such derivatives may be naturally occurring or synthetic derivatives. A “pharmaceutically acceptable derivative or prodrug” means any pharmaceutically acceptable salt, ester, salt of an ester, or other derivative of a compound of this invention which, upon administration to a recipient, is capable of providing (directly or indirectly) a compound of this invention. Particularly favored derivatives and prodrugs are those that increase the bioavailability of the compounds of this invention when such compounds are administered to a mammal (e.g., by allowing an orally administered compound to be more readily absorbed into the blood) or which enhance delivery of the parent compound to a biological compartment (e.g., the skin) relative to the parent species. Preferred prodrugs include derivatives where a group which enhances aqueous solubility or active transport through the gut membrane is appended to the structure of formulae described herein. See, e.g., Alexander, J. et al. Journal of Medicinal Chemistry 1988, 31, 318-322; Bundgaard, H. Design of Prodrugs; Elsevier: Amsterdam, 1985; pp 1-92; Bundgaard, H.; Nielsen, N. M. Journal of Medicinal Chemistry 1987, 30, 451-454; Bundgaard, H. A Textbook of Drug Design and Development; Harwood Academic Publ.: Switzerland, 1991; pp 113-191; Digenis, G. A. et al. Handbook of Experimental Pharmacology 1975, 28, 86-112; Friis, G. J.; Bundgaard, H. A Textbook of Drug Design and Development; 2 ed.; Overseas Publ.: Amsterdam, 1996; pp 351-385; Pitman, I. H. Medicinal Research Reviews 1981, 1, 189-214; Sinkula, A. A.; Yalkowsky. Journal of Pharmaceutical Sciences 1975, 64, 181-210; Verbiscar, A. J.; Abood, L. G Journal of Medicinal Chemistry 1970, 13, 1176-1179; Stella, V. J.; Himmelstein, K. J. Journal of Medicinal Chemistry 1980, 23, 1275-1282; Bodor, N.; Kaminski, J. J. Annual Reports in Medicinal Chemistry 1987, 22, 303-313.

The compounds of this invention may be modified by appending appropriate functionalities to enhance selective biological properties. Such modifications are known in the art and include those which increase biological penetration into a given biological compartment (e.g., epidermis), increase oral availability, increase solubility to allow administration by injection, alter metabolism and alter rate of excretion.

### Cosmetic and Dermatologic Formulations

Compositions of the invention, which include hop extracts, such as xanthohumol and xanthohumol/cyclodextrin complexes, are useful not only for promoting the health and condition of skin, but also for enhancing the appearance of skin. Accordingly, the invention provides for cosmetic and dermatological preparations of various forms. Preferably, the present invention provides personal care compositions that include an effective amount of a combination of the invention, with the balance of the composition comprising one or more compounds from the group of carriers, excipients, liposomes, active ingredients, biological or botanical products, humectants, emollients, surfactants, thickening agents, silicone components, organic sunscreens, preservatives, neutralizing agents, perfumes or pigments. Such compositions improve or enhance skin health, condition, or appearance, and are provided, for example, as a solution, an anhydrous preparation, an oil-free preparation, an emulsion or microemulsion, a gel, a solid stick, an ointment, or as an aerosol. It may also be advantageous to administer compositions of the invention in encapsulated form, for example in collagen matrices or in other conventional encapsulation materials, for example as cellulose encapsulations, in gelatine, wax matrices, or liposomally encapsulated.

In one approach, compositions of the invention are provided in a personal care composition, such as a moisturizing body wash, body wash, antimicrobial cleanser, skin protective cream, body lotion, facial cream, moisturizing cream, facial cleansing emulsion, surfactant-based facial cleanser, facial exfoliating gel, anti-acne treatment, facial toner, exfoliating cream, facial mask, after-shave balm or radioprotective. Skin care compositions include topically applied over-the-counter compositions, anti-fungal treatments, anti-acne treatments, skin protectants, and antiperspirants.

Alternatively, a personal care composition is provided as a makeup composition. Preferred makeup compositions include eye gels, high-melting point lipsticks, lipsticks, lip glosses, lip balms, mascaras, eyeliners, pressed powder formulations, and foundations. If desired, the skin care composition includes a radioprotective agent, such as a sunscreen (e.g., a non-water-resistant sunscreen, water-resistant sunscreen, and water-in-silicone sunscreen).

The invention also provides methods for making personal care compositions comprising combining an effective amount of a combination of the invention with a physiologically acceptable carrier or excipient to provide a personal care composition.

The type of carrier utilized in the present invention depends on the type of product form desired for the personal care composition. In some embodiments, the carrier is a solid, while in other embodiments, it is semi-solid or liquid. Suitable carriers include liquids, as well as semi-solids (e.g., creams, lotions, gels, sticks, ointments, pastes, sprays and mousses). In particular, carriers that are lotions, creams or gels are useful for the topical application of a combination of the invention. The carrier itself may be inert or may possess dermatological benefits of its own. Preferably, the carrier is physically and chemically compatible with a combination of the invention described herein, and does not unduly impair stability, efficacy or other use benefits associated with the compositions of the present invention.

### Nutraceutical Formulations

Nutraceutical compositions are preparations that include compounds of the invention in combination with natural ingredients and supplements that promote good health. Preferably, such nutraceuticals are useful as chemopreventive or chemotherapeutics based on their activity in enhancing health. The combinations provided by the invention contain xanthohumol/cyclodextrin complexes. Information about numerous plants and herbs that have been used to prepare nutraceutical compositions has been compiled and is available in publications including the German Commission E Monographs (by German Federal Institute for Drugs and Medical Devices Commission E), Botanical Safety Handbook Guide for Safe Use and Labeling for Herbs in Commerce, editor M. McGuffin, and HerbalGram, a quarterly publication of the American Botanical Council, which references numerous clinical trials that have been performed using nutraceuticals.

The actions of nutraceutical compositions may be fast or/and short-term or may help achieve long-term health objectives. Nutraceutical compositions may comprise dried and ground plant (e.g., hops) tissue or extracts from these tissues in an acceptable medium as a natural approach for treatment or prevention of a disease described herein. The nutraceutical compositions may be contained in an edible material, for example, as a dietary supplement or a pharmaceutical formulation. As a dietary supplement, additional nutrients, such as vitamins, minerals or amino acids may be included. The composition can also be a drink or a food product, e.g., tea, soft drink, juice, milk, coffee, cookie, cereal, chocolate, and snack bar. If desired, the composition can be sweetened by adding a sweetener such as sorbitol, maltitol, hydrogenated glucose syrup and hydrogenated starch hydrolyzate, high fructose corn syrup, cane sugar, beet sugar, pectin, or sucralose.

In another example, the composition of this invention, containing an edible carrier, is a component of a food product (e.g., yogurt, milk, or soy milk) or a food supplement (e.g., a nutrient supply or an herbal product). Examples of an edible carrier include starch, cyclodextrin, maltodextrin, methylcellulose, carbonmethoxy cellulose, xanthan gum, and aqueous solutions thereof. Such food products can be prepared by methods well known in the food industry. As used herein, the term “food” broadly refers to any kinds of liquid and solid/semi-solid materials that are used for nourishing humans and animals, for sustaining normal or accelerated growth, or for maintaining stamina or alertness.

Composition of this invention, containing xanthohumol, can be used to treat a variety of skin diseases and disorders.

A nutraceutical comprising a composition of this invention can be in the form of a solution. Typically, the composition of the invention is provided in a medium, such as a buffer, a solvent, a diluent, an inert carrier, an oil, or a crème. In one embodiment, the composition is present in an aqueous solution that optionally contains a non-aqueous co-solvent, such as an alcohol. The composition can also be in the form of powder, paste, jelly, capsule, or tablet. Lactose and corn starch are commonly used as diluents for capsules and as carriers for tablets. Lubricating agents, such as magnesium stearate, are typically added to form tablets.

### Topical Administration

Topical administration of the compositions of this invention is especially useful for preventing or treating a skin disease or disorder or for enhancing the appearance of skin. The xanthohumol-containing topical formulations described above can further include another active agent, such as a vitamin (e.g., vitamin B, 1,25-dihydroxy vitamin D3, vitamin K, vitamin A, and vitamin C), an anti-microbial agent (e.g., tolnaftate, ketoconazole, erythromycin, and tetracycline), an insect-repellent (e.g., aliphatic, cyclic or aromatic amides, citronella oil, terpineol, cineole, neem oil, and ethyl butyacetylaminopropionate), a self-tanning agent (e.g., dihydroacetone and lawsone), an anti-inflammatory agent (e.g., hydrocortisone, prednisone, prednisolone, aspirin, aloe vera, and mixtures thereof), a topical analgesics (e.g., lidocaine, benzocaine, butacaine, and clove oil), and/or a skin redness reducer (e.g., guanidine derivatives or L-arginine derivatives).

For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water.

### Subject Monitoring

The disease state or treatment of a subject having a skin disease or disorder can be monitored during treatment with a composition of the invention. Such monitoring may be useful, for example, in assessing the efficacy of a particular agent in a patient. Therapeutics that promote skin health or that enhance the appearance of skin are taken as particularly useful in the invention.

### Kits

The invention provides kits for the treatment or prevention of a skin disease or disorder, or symptoms thereof. In one embodiment, the kit includes a pharmaceutical pack comprising an effective amount of xanthohumol. Preferably, the compositions are present in unit dosage form. In some embodiments, the kit comprises a sterile container which contains a therapeutic or prophylactic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired compositions of the invention or combinations thereof are provided together with instructions for administering them to a subject having or at risk of developing a skin disease or disorder. The instructions will generally include information about the use of the compounds for the treatment or prevention of a skin disease or disorder. In other embodiments, the instructions include at least one of the following: description of the compound or combination of compounds; dosage schedule and administration for treatment of a skin disease symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

The following examples are provided to illustrate the invention, not to limit it. Those skilled in the art will understand that the specific constructions provided below may be changed in numerous ways, consistent with the above described invention while retaining the critical properties of the compounds or combinations thereof.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Xanthohumol Inhibits Acne-Related Bacteria

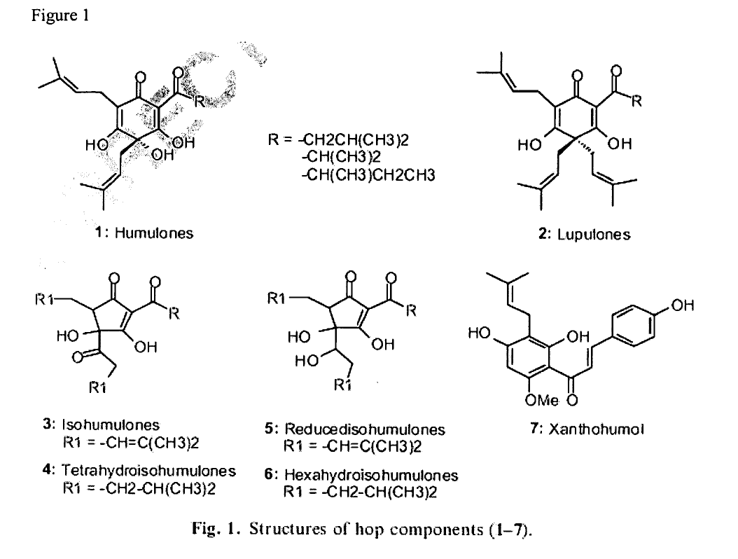

Dried hop flowers (hops) have attracted a great deal of attention as a source of small molecules, such as humulones, lupulones, isohumulones and xanthohumol (FIG. 1) with potential for beneficial effects on health. The prior art has not shown that hops have in vitro biological activity against acne vulgaris. The present studies address the effects of hop components on biological factors involved in the pathogenesis of acne vulgaris.

The antibacterial effects of seven hops components against five different strains of bacteria involved in primary or secondary skin and soft tissue infections was evaluated, with broth dilution methods. Results of this analysis are shown at Table 1. The lowest MIC values against P. acnes and S. pyogenes were observed for lupulones 2, the values having reached 0.1 and 0.3 mg/ml, respectively (Table 1).

TABLE 1
Antibacterial activities (MIC^{a} and MBC^{b}) of hop components against the
most common bacteria causing primary or secondary skin or soft tissue
infections

| | MIC (g/mL) | | | | |
|---|---|---|---|---|---|
| | [MBC (g/mL)] | | | | |
| Conpound | P. acnes | S. aureus | S. epidermidis | S. pyogenes | K. rhizophila |
| Humulones (1) | 10 | 10 | 10 | 3 | 30 |
| | [30] | [100] | [100] | [100] | [100] |
| Lupulones (2) | 0.1 | 1 | 10 | 0.3 | 1 |
| | [0.3] | [100] | [10] | [30] | [10] |
| Isohumulones (3) | 30 | 10 | 30 | 10 | 100 |
| | [>100] | [>100] | [>100] | [>100] | [100] |
| Reduced isohumolones (5) | 10 | 3 | 10 | 10 | ND |
| | [>100] | [>100] | [>100] | [>100] | [ND] |
| Tetrahydro-isohumulones (4) | 3 | 3 | 10 | 10 | ND |
| | [10] | [>100] | [100] | [100] | [ND] |
| Hexahydro isohumulones (6) | 3 | 10 | 10 | 3 | ND |
| | [100] | [>100] | [100] | [>100] | [ND] |
| Xanthohumol (7) | 3 | 1 | 3 | 1 | 1 |
| | [3] | [100] | [10] | [3] | [10] |

ND: Not Determined
^{a}MIC indicates minimal 100% inhibitory concentration.
^{b}MBC indicates the lowest concentration required to kill an organism.

The lowest MIC values against S. epidermidis, K. rhizophila, and S. aureus were observed for lupulones 2 and xanthohumol 7, the value having reached 1 mg/ml. It is also important that all the strains are sensitive not only to naturally occurring hop components 1, 2, and 7, but also the chemically modified ones, 3, 4, and 5. Such strong inhibitory activities of lupulones 2 and xanthohumol 7 against acne-related bacteria have not been reported among natural products derived from edible plants. The low MIC values are comparable to the most commonly prescribed antibiotics for topical acne treatment (e.g., clindamycin and erythromycin). Interestingly, lupulones 2 and xanthohumol 7 exhibited bactericidal activity against P. acnes and the MBC values were 0.3 and 3.0 mg/ml, respectively (Table 1).

### Example 2

### Xanthohumol has Anti-Collagenase Activity

Results of activity against interstitial collagenase (MMP-1) and neutrophil collagenase (MMP-8) are listed in Table 2.

TABLE 2
Anticollagenase activities^{a} of hops against MMP-1 and MMP-8
involved in acne pathogenesis

| | Concentration | Inhibitory ratio (%) | |
|---|---|---|---|
| Compound | (μg/mL) | MMP-1 | MMP-8 |
| Humulones (1) | 100 | 62 | 62 |
| | 30 | 11 | 29 |
| Lupulones (2) | 100 | 27 | 28 |
| | 30 | 6 | 12 |
| Isohumulones (3) | 100 | 0 | 0 |
| Reduced isohumulones (5) | 100 | 0 | 0 |
| Tetrahydroisohumulones (4) | 100 | 0 | 0 |
| Hexahydroisohumulones (6) | 100 | 0 | 0 |
| Xanthohumol (7) | 100 | 91 | 99 |
| | 30 | 65 | 70 |
| | 10 | 25 | 31 |

^{a}Data represent the mean of three independent measurements

Xanthohumol 7 showed the highest activity against both collagenases; the IC₅₀ values were 20.5 and 16.8 mg/ml, respectively, whereas humulones 1 and lupulones 2 showed only weak activity with 27% and 28% inhibition for MMP-1, and 61% and 62% for MMP-8, respectively. Chemically modified derivatives 3, 4, 5, and 6 did not show activity up to a concentration of 100 mg/ml.

Extracellular proteases, in particular MMPs, have been implicated in a number of dermatological conditions; for example, in chronological aging, inflammatory matrix remodeling, and hyperproliferative skin disorders (Choi et al. 2008 J. Invest. Dermatol. 128: 846-854). These processes involve the increased breakdown of various components of the extracellular matrix in the skin, notably collagen, elastin, and fibronectin. Enhanced expression of the collagenases MMP-1 and -8 has been described as playing a central role in connective type-1 collagen

breakdown in the skin (Brenneisen et al. 2002 Ann. NY Acad. Sci. 973: 31-43). The transcription factors nuclear factor κ-B (NFkappaB) and activator protein-1 (AP-1) are activated in acne lesions with consequent elevated expression of their target gene products, inflammatory cytokines and matrix-degrading metalloproteinases, respectively. These elevated gene products are molecular mediators of inflammation and collagen degradation in acne lesions in vivo. Recently, this new knowledge has enabled a rational strategy for the development of drugs that can target the inflammation and matrix remodeling that occurs in severe acne (Kang et al. 2005 Am. J. Pathol. 166: 1691-1699).

### Example 3

### Xanthohumol has Antioxidant Activity

It is well documented that inflammation caused by oxidative damage has been implicated in not only in skin disorders, but also in various systemic chronic diseases, such as cancer, Alzheimer's disease, rheumatoid arthritis, cardiovascular disease, cataracts, and other ageing processes. Reactive oxygen species are essential intermediates in oxidative metabolism. Nonetheless, when generated in excess, ROS in various active forms can damage tissues. The radical-scavenging activity of hop components 1 and 2 has been evaluated previously using a conventional 1,1-Diphenyl-2-picrylhydrazyl (DPPH) assay, employing one of the stable nitrogen-centered free radicals. These components have promise for their antioxidant potential. Xanthohumol was also shown to scavenge hydroxyl and peroxyl radicals, and superoxide anion radicals, in an oxygen radical absorbance capacity (ORAC) assay (Gerhauser 2005 Mol. Nutr. Food Res. 49: 827-831; Vogel et al. 2008 Bioorg. Med. Chem. 16: 4286-4293).

A standardized ORAC value shows the scavenging capacity of antioxidants against the peroxyl radicals, which are among the most common ROS found in the body. Thus the ORAC method has become a widely used method for assessing antioxidant capacity in biological samples and foods. However, because of its inability to measure both hydrophilic and lipophilic antioxidants, the method has its limitations.

An ORAC method for lipophilic antioxidants was further developed and validated using fluorescein as the fluorescent probe (Prior et al. 2003 J. Agric. Food Chem. 51: 3273-3279). This method has the advantage that similar assay conditions and standards can be used for both hydrophilic and lipophilic antioxidant components, such that the two values can be added together to obtain a total antioxidant capacity.

Table 3 shows six assays for the antioxidant capacity of hop components against various ROS formed in human skin (Bickers and Athar 2006 J. Invest. Dermatol. 126: 2565-2575).

TABLE 3
Reactive oxygen species (ROS) related to inflammation

| | ROS | Assay |
|---|---|---|
| 1 | Peroxyl nitrite | NORAC |
| 2 | Hydroxyl radical | HORAC |
| 3 | Peroxyl radical, fat-soluble | ORAC-L |
| 4 | Peroxyl radical, water-soluble | ORAC-H |
| 5 | Superoxide | SOD |
| 6 | Heavy metal cation Fe³⁺ | FRAP |
| 7 | Singlet oxygen ¹O₂. | SOAC |

ORAC-H: ORAC_{hydro}, water-soluble antioxidant capacity
ORAC-L: ORACₗᵢₚₒ, lipid soluble antioxidant capacity

In the present work, the ORAC value is expressed as the sum of values of both the water-soluble and fat-soluble parts. Green tea catechins (Polyphenon 60) were used as the control with the highest ORAC value among edible plants. Vitamin C and vitamin E were used as controls for water- and fat-soluble molecules, respectively.

The results of these studies are shown in Table 4.

TABLE 4
Oxygen radical absorbance capacity (ORAC) of hop components

| | ORAC-H | ORAC-L | ORAC-Total |
|---|---|---|---|
| | (Trolox | (Trolox | (Trolox |
| Conpound | equivalent) | equivalent) | equivalent) |
| Humulones (1) | 0.62 | 0.60 | 1.2 |
| Lupulones (2) | 0.84 | 1.1 | 1.9 |
| Isohumulones (3) | 0.41 | 0.054 | 0.46 |
| Reduced isohumolones (5) | 0.64 | 0.062 | 0.65 |
| Tetrahydro-isohumulones (4) | 0.034 | 0.011 | 0.045 |
| Xanthohumol (7) | 2.1 | 2.1 | 4.2 |
| Vitamin C | 1.4 | — | 1.4 |
| Vitamin E | — | 0.75 | 0.75 |
| Polyphenon 60 | 4.2 | 0.006 | 4.2 |

ORAC-Total: ORACₗᵢₚₒ + ORAC_{hydro}
No activity detected

The higher the total ORAC score, the higher the antioxidant capacity. The total ORAC value of xanthohumol 7 was comparable to that of Polyphenon 60, and much higher than that of vitamins C and E. In addition xanthohumol 7 shows equal activity in both fat- and water-soluble antioxidant capacity. These data indicate that xanthohumol 7 may serve as a well-balanced antioxidant similar to the combination of vitamins C and E. Taken altogether, the antioxidant properties of xanthohumol 7 deserve attention.

### Example 4

### Xanthohumol has Singlet Oxygen-Quenching Activity

Table 5 shows the singlet oxygen-quenching activity of hop components.

TABLE 5
Singlet oxygen absorbance capacity (SOAC) of hop components

| | SOAC |
|---|---|
| Conpound | (Vitamin E equivalent) |
| Humulones (1) | 0.75 |
| Lupulones (2) | 0.80 |
| Isohumulones (3) | 0.40 |
| Reduced isohumolones (5) | 0.55 |
| Tetrahydro-isohumulones (4) | 0.59 |
| Xanthohumol (7) | 14.1 |
| Vitamin E | 1.0 |
| Polyphenon 60 | 1.8 |

Alpha-tocopherol (vitamin E) was used as the calibration standard, and the SOAC result is expressed as mmol VtE/g. The higher the SOAC score, the higher the singlet oxygen-quenching capacity. Interestingly, the xanthohumol SOAC value was about 8-15 times higher than that of vitamin E or Polyphenon 60. Singlet oxygen has been postulated to be a highly reactive and toxic intermediate against skin. It is known that the acne-causing bacterium P. acnes naturally produces high amounts of intracellular porphyrins, mostly coproporphyrin (Ashkenazi et al. 2003). Singlet oxygen can be generated on the skin surface from P. acnes porphyrins under sunlight and induce serious skin damage (Arakane et al. 1996 Biochem. Biophys. Res. Commun. 223: 578-582). Various skin disorders progress through a singlet oxygen-dependent mechanism, including acne, atopic dermatitis, and skin aging.

Squalene is also a component of acne sebum. Both porphyrins and squalene are directly exposed to the external environment and play a key role in skin physiology. Recently it was demonstrated that squalene peroxidation during solar exposure is mainly caused by singlet oxygen and by free radical attack, suggesting that sun skin-care cosmetics should make use not only of free radical scavengers but also of singlet oxygen quenchers (Auffray 2007 Int. J. Cosmet. Sci. 29: 23-29). Xanthohumol's singlet oxygen-quenching activity might be widely used to develop new therapeutic medications for immune and inflammatory diseases (Moan and Juzenas 2006 J. Environ. Pathol. Toxico. Oncol. 25: 29-50)

Recently many scientific papers have addressed the relationship between lifestyle diseases and chemical attacks by singlet oxygen and free radicals. As evidenced by the results reported herein, xanthohumol is among the most powerful naturally occurring antioxidants in terms of singlet oxygen-quenching activity. The results provided herein indicate that xanthohumol's singlet oxygen-quenching activity comes close to that of β-carotene, a precursor of retinoids.

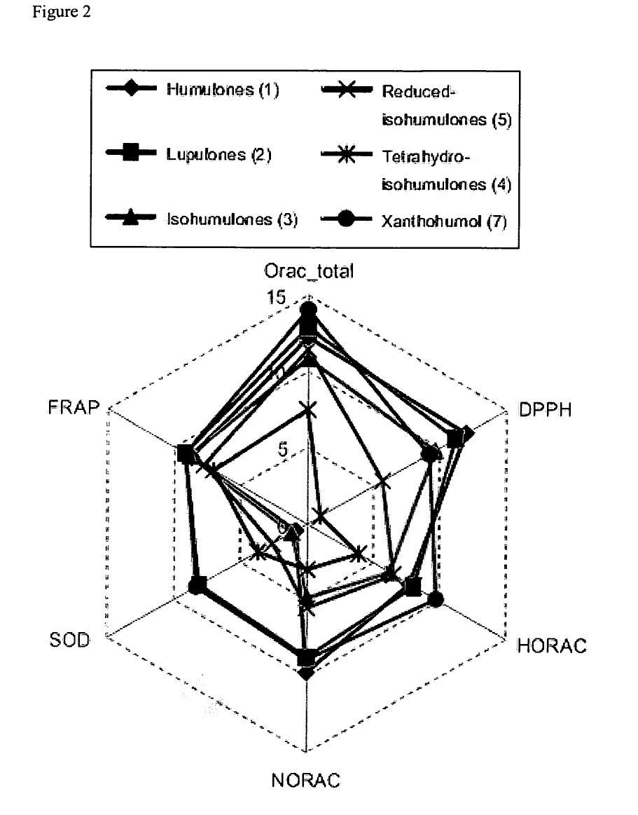

The radar chart in FIG. 2 shows how the hop components ranked in the six antioxidant assays described herein. The SOAC scores are not included in this figure; ORAC values used are the sum of lipophilic and hydrophilic results. For each assay category, scaling was made logarithmic so that the plot on the chart resembles the associated rating. The chart graphically shows areas of relative strength and relative weakness, as well as depicting the general overall antioxidative capacity. The larger the spatial area, the higher the overall antioxidative capacity. Significantly, xanthohumol 7 (green) shows the highest quenching activity against peroxyl radical (ORAC-total), superoxide (SOD), ferric ion (FRAP), and hydroxyl radical (HORAC). Humulones 1 (yellow) show the strongest quenching activity against the nitrite radical (NORAC) and DPPH.

On the other hand, chemically reduced derivatives 3, 4, and 5 show poor antioxidant capacity in comparison to natural compounds. As active ingredients with anti-inflammatory activity due to their improved chemical stability and ease of handling, chemically reduced hop derivatives 4, 5, and 6 are used for relief of arthritis symptoms. The chemical modifications significantly impair one of the most important biological properties essential for anti-inflammatory efficacy, antioxidant capacity (Altindag et al. 2007 Rheumatol Int. 27:339-344).

In this study, biological assays were used to explore how hops extracts alter three activities involved in acne pathogenesis: bacterial proliferation, increased sebum production caused by reactive oxygen species, particularly singlet oxygen, and excessive matrix remodeling through type-1 collagen breakdown by MMPs.

Over-the-counter and other nonprescription medications are often more effective when they modulate two or more of these activities when treating acne. As a result, dermatologists have generally concluded that taking a more comprehensive approach gives better results. Thus the recommendations given to patients frequently suggest using more than one agent at a time. Unfortunately, mixing medications does not always work because of unwanted interactions that lead to a decrease in or loss of efficacy.

Results reported here confirm that xanthohumol 7 and lupulones 2 demonstrate multiple activities against the main biological events responsible for the pathogenesis of acne. It is clear that the anti-inflammatory activity of naturally occurring hop components inhibits the proinflammatory mediators (e.g., COX-2, PGE2, NO, NFkappaB, IL-1β, and TNF-α) responsible for skin inflammation. Thus, the unique multifunctional activities of lupulones 2 and xanthohumol 7 among hop components, could prove to be of value for future human clinical studies in comparison with commercial combinations of synthetic drugs.

### Example 5

### Preparation of Xanthohumol-Cyclodextrin Complex

Xanohop A (a hop extract containing 50% by weight xanthohumol) was dissolved in ethanol (1.25 g Xanohop A per 5 ml ethanol) to form a xanthohumol solution, which was then mixed with an aqueous solution containing 10% gamma cyclodextrin or beta cyclodextrin to form a mixture. The pH of the mixture was adjusted to 10.9 to 11.0. After removing some brown precipitates via filtration, H₂SO₄ was added to the mixture such that it pH was re-adjusted to 7.9. The mixture was then kept still at room temperature overnight to allow formation of yellowish precipitates. The precipitates were collected by filtration and dried to produce 4.52 g powder, which contained 12% by weight xanthohumol. In the powder, cubic crystals of xanthohumol-gamma cyclodextrin complex or xanthohumol-beta cyclodextrin complex were observed under optical microscope. Note that pure xanthohumol forms needle-shaped crystals. The iso-xanthohumol/xanthohumol ratio in the resultant powder was 0.015.

As shown below, the water solubility of xanthohumol contained in the xanthohumol-gamma cyclodextrin and xanthohumol-beta cyclodextrin complexes thus prepared was much higher than pure xanthohumol and xanthohumol in Xanohop A.

Pure xanthohumol (XN), Xanohop A, xanthohumol-gamma cyclodextrin (XN/gCD), and xanthohumol-beta cyclodextrin (XN/bCD) were dispersed in water at a ratio of 1 mg xanthohumol per ml of water. After being sonicated for 5 minutes, the resultant suspensions were centrifuged at 3,000 rpm for 2 minutes or 12,000 rpm for 3 minutes. The supernatants thus formed were collected, diluted (1:10) with methanol, and then injected into HPLC to determine xanthohumol concentrations. Results thus obtained indicated that xanthohumol in XN/gCD complex is about 20 times more soluble in water than pure xanthohumol and about 10 times more soluble in water than Xanohop A. The water solubility of XN/bCD complex is even higher than that of XN/gCD. See Table 6 below:

TABLE 6
Water-Solubility of Xanthohumol (XN)

| | Water-solubility of | Water-solubility of |
|---|---|---|
| | XN(3,000 rpm) | XN (12,000 rpm) |
| Pure XN | 4 ug/ml | 2 ug/ml |
| Xanohop A | 12 ug/ml | 6 ug/ml |
| XN/gCD | 78 ug/ml | 45 ug/ml |
| XN/bCD | 330 | 180 |

Xanohop A (sample 1), XN-cremophore composition (sample 2), and XN/gCD complex (sample 3) were tested for conversion of xanthohumol to isothohumol as follows. Samples 1, 2, and 3 were incubated at 75° C. for 5 days (equivalent to one-month storage at room temperature). The contents of iso-xanthohumol in these samples were determined before and after the incubation.

Before incubation, the contents of iso-xanthohumol (versus 100 mg xanthohumol) in Samples (1) and (2) are 6.5 mg and 7.0 mg, respectively, and that in Sample (3) is only 1.2 mg. After incubation, the contents of iso-xanthohumol in Samples (1), (2), and (3) are 8.5 mg, 28.5 mg, and 2.0 mg, respectively. These results indicate that Sample (3), i.e., xanthohumol-gamma cyclodextrin complex, contains the lowest isoxanhumol contents both before and after one-month storage, compared to Sample (1), i.e., Xanohop A, and Sample (2), i.e., xanthohumol-cremophore composition.

### Example 6

### Using Aqueous Lotion Containing Xanthohumol-Cyclodextrin Complex for Treating Acne

A xanthohumol-cyclodextrin complex of xanthohumol and beta-cyclodextrin is mixed with an aqueous alcohol solution to form an aqueous lotion containing 0.01 g/L by weight xanthohumol. This aqueous lotion is topically applied to acnes in patients, twice a day, for two weeks. Its anti-acne effect is then examined by routine medical procedures. In other embodiments, a topical formulation of the invention comprises at least about 0.001%, 0.005%, 0.01%, 0.05%, 0.1%, 0.5%, 1%, 2%, 3%, 4% or 5% xanthohumol or a xanthohumol-cyclodextrin complex.

### Hop Components

With reference to FIG. 1, three compounds, humulones 1, lupulones 2 and xanthohumol 7 are naturally occurring hop components. Isohumulones 3 are isomerized molecules that are converted from humulones 1. Other molecules, tetrahydroisohumulone 4, reduced isohumulone 5, and hexahydroisohumulone 6, are hydrogenated derivatives of isohumulones 3 with improved stability. The humulones-rich fraction (containing 34% of humulones 1; calcd for C₂₁H₃₀O₆) was produced by fractionation of a CO₂ extract (obtained from John I Haas Inc., Yakima, Wash.) followed by purification through a column packed with Amberlite 20 FPX66 (food grade), with alkaline water used as an eluent aqueous at pH 10. The lupulones-rich fraction (containing 10% of lupulones 2; calcd for C₂₆H₃₈O₄) was produced by fractionation of the CO₂ extract, followed by purification through active charcoal treatment in an aqueous solution at pH 10 (U.S. application Ser. No. 11/868,226).

Isohumulones 3 (calcd for C₂₁H₃₀O₅, 10% aqueous solution) converted by treatment of humulones 1 at 70° C. under alkaline conditions, were also obtained from John I Haas Inc. Reduced isohumolones 5 (calcd for C₂₁H₃₂O₅), synthesized by reduction of isohumulones 3 with sodium borohydride (NaBH₄) in water at pH 10, were likewise obtained from John I Haas Inc. Similarly obtained were tetrahydro- and hexahydroisohumulones 4 (calcd for C₂₁H₃₂O₅) and -6. (calcd for C₂₁H₃₄O₅), prepared by hydrogenation of isohumulones 3, and reduced isohumulones 5, respectively, in the presence of 5% palladium on charcoal (Pd/C) in water at pH 10.

A xanthohumol-rich fraction (containing 50% of xanthohumol 7, calcd for C₂₁H₂₂O₅) was prepared by extraction of spent hops (obtained from Nateco2, Wolnzach, Germany) with acetone, and subsequent purification by a pH-adjusted salting-out method employing aqueous ethanol in the presence of NaCl.

### Antimicrobial Assays

Antimicrobial susceptibility testing was performed using a broth dilution method. Gentamicin or ampicillin was used as a control to verify the methodology. A stock solution (20 mg/ml) of the test substance (or vehicle control) was prepared in dimethyl sulfoxide. Serial dilution was done in microwell plates. Reinforced Clostridial Medium was used for P. acnes (ATCC 6919). Three hundreds ml of the test substance was added to the test tube containing P. acnes (10⁵ CFU/ml) in 2.7 ml of cultures grown under anaerobic conditions. Mueller-Hinston Broth suitable for culturing S. epidermidis (ATCC 12228), K. rhizophila (formerly M. luteus) (ATCC 9341), and S. aureus (ATCC 6538P), and Brain Heart Infusion Broth suitable for culturing S. pyogenes (ATCC 14289) were used. A total of 100 ml of the test substance was added to the test tube containing the other microorganisms (10⁵CFU/ml) in 0.9 ml of cultures grown under controlled conditions. After 2 days for acnes and 1 day for the others, growth of the culture was examined and scored positive for inhibition of growth, or negative for no effect upon growth. Samples from those tubes that scored positive was plated onto an agar plate and incubated under controlled conditions. The minimum inhibitory concentration (MIC) was defined as the lowest concentration that resulted in no visible growth after 2 days for P. acnes or 1 day for the others. The minimum bactericidal concentration (MBC) was defined as the lowest concentration at which the microorganisms failed to grow in each medium and on each agar plate.

### Antioxidant Assays

Both hydrophilic and lipophilic oxygen radical absorbance capacity (ORAC) assays were carried out at Brunswick Laboratory (Wareham, Mass.) based on the modified ORACfl method reported by Ou et al. (2001 J. Agric. Food Chem. 49: 4619-4626, 2002 J. Agr. Food Chem. 50: 3122-3128). Trolox, a water-soluble vitamin E analog, was used as the calibration standard. The data are expressed as mmole of Trolox equivalents per gram (mmol TE/g). The acceptable precision of the ORAC assay is a 15% relative standard deviation. Caffeic acid was used as the calibration standard. Hydroxyl radical ORAC (HOR-AC) is expressed as mmole caffeic acid equivalent per gram (mmol CAE/g). Trolox was used as the calibration standard. The peroxyl nitrite ORAC (NORAC) result is expressed as mmol TE/g. Alpha-tocopherol (vitamin E) was used as the calibration standard, and the singlet oxygen absorbance capacity (SOAC) result is expressed as mmole a-tocopherol equivalent (mmol VtE) per gram (Aubry et al. 1989). The abbreviation for the 1,1-diphenyl-2-picrylhydrazyl radical is DPPH. Radical-scavenging activity was measured by the change in absorbance at 517 nm with the DPPH result expressed as mmol TE/g. Similarly, FRAP is an abbreviation for the ferric reducing antioxidative power method, which utilizes chemical conversion of the yellow Fe 3+-2,4,6-tripyridyl s-triazine (TPTZ) complex to the blue Fe 2+-TPTZ complex by electron donation under acidic conditions (Okada and Okada 1998 J. Agric. Food Chem. 46: 401-406). The FRAP result is expressed as mmol TE/g (Ou et al. 2002b). Superoxide dismutase (SOD) was used as a calibration standard; the SOD result is expressed as kilo unit SOD equivalent (kunitSODeq) per gram.

### Anticollagenase Assays

Human recombinant matrix metalloproteinase-MMP-1) pro-enzyme, expressed in mammalian cells, and human nutrophil MMP-8 pro-enzyme were activated with 4-aminophenylmercuric acetate for 60 min 37 1 C, respectively (Knight et al. 1992 FEBS Lett. 296: 263-266). Hop components and vehicle were preincubated with 5 nM MMP- and 6 nM MMP-8 active enzymes in a modified MOPS buffer, pH 7.2, for 60 minutes at 37 C. The reaction was initiated by addition of 4 mM Mca-Pro-Leu-Gly-Leu-Dpa-Ala-Arg for another 120-minute incubation period. The determination of the amount of Mca-Pro-Leu-Gly formed was read spectrofluorimetrically at 340/400 nm. Tissue inhibitors of metalloproteinase TIMP-1 and TIMP-2 were used as positive controls (Olson et 1997 J. Biol. Chem. 272: 29975-29983).

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A composition for treating or preventing a skin disease, disorder, or condition the composition comprising an effective amount of a xanthohumol/cyclodextrin complex in a pharmaceutically acceptable excipient.

2. The composition of claim 1, wherein the skin disease, disorder or condition is selected from the group consisting of acne, atopic dermatitis, contact dermatitis, eczema, rosacea, seborrhea, psoriasis, wrinkles, skin thinning, surface blood vessels, loss of elasticity, hyperpigmentation, photodamage, aging, and loss of subcutaneous fat layer.

3. A composition for enhancing the appearance of skin, the composition comprising an effective amount of a xanthohumol or a xanthohumol/cyclodextrin complex in a pharmaceutically acceptable excipient.

4. (canceled)

5. The composition of claim 1, wherein the composition has anti-collagenase activity.

6. The composition of claim 5, wherein the composition enhances collagen synthesis or deposition in the cell.

7. (canceled)

8. (canceled)

9. (canceled)

10. The composition of claim 1, wherein the composition is formulated for topical administration.

11. The composition of claim 1, wherein an effective amount is 0.5 ug to 200 mg per dose per day.

12. (canceled)

13. (canceled)

14. (canceled)

15. (canceled)

16. (canceled)

17. (canceled)

18. (canceled)

19. (canceled)

20. The composition of claim 1, wherein the xanthohumol is 3′-[3,3-dimethyl allyl]-2′,4′,4-trihydroxy-6′-methoxychalcone) or a prenylated chalcone derived from hops.

21. The composition of claim 1, wherein the xanthohumol is selected from the group consisting of xanthoangelol, xanthoangelol F, 4-hydroxyderricin, 4-O-methylxanthohumol, isobavachalcone, xanthoangelol H, xanthogalenol, desmethoxyxanthohumol, 5′-prenylxanthohumol, tetrahydroxanthohumol, 2′,4′,6′,4-terahydroxy-3′-C-geranylchalcone, dehydrocycloxanthohumol, 4-O-5′-C-diphenylxanthohumol, 4′-O-methylxanthohumol, and a xanthohumol metabolite.

22. The composition of claim 1, wherein the cyclodextrin is alpha-cyclodextrin, beta-cyclodextrin, or gamma-cyclodextrin.

23. The composition of claim 1, wherein the cyclodextrin compound is gamma-cyclodextrin.

24. The composition of claim 1, wherein the cyclodextrin is selected from the group consisting of hydroxypropyl-beta-cyclodextrin, sulfobutyl ether-beta-cyclodextrin, heptakis(2,6-di-O-methyl)-beta cyclodextrin, C₁₋₂₄-alkyl-gamma-cyclodextrin, and C₁₋₂₄-hydroxyalkyl-gamma-cyclodextrin.

25. The composition of claim 1, wherein the xanthohumol and the cyclodextrin have a molar ratio of 2:1 to 1:4 in the complex.

26. The composition of claim 25, wherein the molar ratio is 1:2.

27. The composition of claim 25, wherein the molar ratio is 1:3.

28. The composition of claim 1, wherein the composition comprises or consists essentially of 0.01-30% by weight xanthohumol.

29. The composition of claim 1, wherein the composition comprises or consists essentially of 0.05-20% by weight xanthohumol.

30. The composition of claim 1, wherein the composition comprises or consists essentially of 0.1-10% by weight xanthohumol.

31. The composition of claim 1, wherein the xanthohumol/cyclodextrin complex is at least about 5-10 times more soluble in water than xanthohumol alone.

32. The composition of claim 1, wherein the composition comprises less than about 5% isoxanthohumol.

33. The composition of claim 1, wherein the composition comprises less than about 3-5% isoxanthohumol after 3-6 months storage.

34. The composition of claim 1, wherein the composition is devoid of isoxanthohumol, a cyclodextrin/isoxanthohumol complex or comprises less than about 10% of a cyclodextrin/isoxanthohumol complex.

35. (canceled)

36. (canceled)

37. (canceled)

38. A method for treating or preventing a skin disease, disorder or condition in a subject, the method comprising administering to the subject an effective amount of a xanthohumol/cyclodextrin complex.

39. The method of claim 38, wherein the skin disease, disorder or condition is selected from the group consisting of acne, atopic dermatitis, contact dermatitis, eczema, rosacea, seborrhea, psoriasis, wrinkles, skin thinning, surface blood vessels, loss of elasticity, hyperpigmentation, photodamage, aging, and loss of subcutaneous fat layer.

40. A method for enhancing the appearance of skin in a subject, the method comprising administering to the subject an effective amount of xanthohumol or a xanthohumol/cyclodextrin complex.

41. The method of claim 40, wherein the skin's appearance is enhanced by reducing the appearance of fine lines, wrinkles, sagging, or hyperpigmentation.

42. The method of claim 40, wherein the skin's appearance is enhanced by increasing skin smoothness, firmness, or elasticity.

43. A method for ameliorating acne in a subject in need thereof, the method comprising administering to the subject an effective amount of xanthohumol or a xanthohumol/cyclodextrin complex.

44. The method of claim 43, wherein the method reduces inflammation and matrix remodeling associated with severe acne.

45. The method of claim 43, wherein the method reduces bacteria present on skin, reduces oxidative damage, and reduces inflammation.

46. The method of claim 43, wherein the method reduces the survival or proliferation of a bacteria selected from the group consisting of Propionibacterium acnes, Staphylococcus epidermidis, Staphylococcus aureus, Kocuria rhizophila and Staphylococcus pyogenes.

47. (canceled)

48. (canceled)

49. (canceled)

50. A method of preparing a xanthohumol/cyclodextrin complex comprising
(a) providing a mixture of xanthohumol and gamma cyclodextrin;
(b) adjusting the pH of the mixture to 10-12, thereby providing for xanthohumol/cyclodextrin complex formation;
(c) re-adjusting the pH to 6-9 to allow precipitation of the xanthohumol/cyclodextrin complex; and
(d) recovering the complex.

51. A composition made by the method of claim 50, said composition comprising a xanthohumol/cyclodextrin complex, wherein the composition comprises less than about 5% isoxanthohumol.

52. (canceled)

53. (canceled)
